(19) United States
(12) Patent Application Publication
(10) Pub. No.: US 2025/0266033 A1
(43) Pub. Date: Aug. 21, 2025

(54) DYNAMIC LIST-BASED SPEECH TESTING

(71) Applicant: Cochlear Limited, Macquarie University, NSW (AU)

(72) Inventor: Filiep J Vanpoucke, Huldenberg (BE)

(21) Appl. No.: 18/858,454

(22) PCT Filed: Apr. 26, 2023
(86) PCT No.: PCT/IB2023/054307

§ 371 Date: Oct. 21, 2024

Related U.S. Application Data
(60) Provisional application No. 63/335,457, filed on Apr. 27, 2022.

(51) Int. Cl. G10L 13/08; A61B 5/12; G10L 13/027

(52) U.S. Cl. CPC G10L 13/08; A61B 5/121; G10L 13/027

(57) ABSTRACT

Presented herein are techniques for dynamically generating test materials for implementing speech test to, for example, hearing aid users or users of cochlear implants. Dynamic word list generation techniques are disclosed. Such techniques generate word lists with qualities intended to implement specific speech tests. For example, the disclosed techniques may be used to generate word lists with predetermined phonetic balance and/or perceptual balance. The disclosed techniques also provide for the generation of audio signals associated with the word lists used during the speech tests.

### BACKGROUND

### Field of the Invention

The present invention relates generally to speech testing.

### Related Art

Medical devices have provided a wide range of therapeutic benefits to recipients/users over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In some aspects, the techniques described herein relate to a method that includes compiling a pool of words. A subset of the pool of words with a predetermined phonetic balance is dynamically generated from the pool of words. A speech test is conducted for a subject using the subset of the pool of words.

In other aspects, the techniques described herein relate to a method that includes compiling data indicative of a pool of words with a predetermined phonetic distribution. A search algorithm is applied to the data indicative of the pool of words to generate data indicative of a subset of the pool of words such that a phonetic distribute of the subset of the pool of words is within a threshold of the predetermined phonetic distribution. A test set of words for a speech test for a subject is generated from the data indicative of the subset of the pool of words.

In still other aspects, the techniques described herein relate to a method that includes compiling data indicative of a pool of words with a predetermined phonetic distribution. A search algorithm is applied to the data indicative of the pool of words to generate data indicative of a subset of the pool of words such that a phonetic distribution of the subset of the pool of words is within a threshold of the predetermined phonetic distribution. An audio signal is generated for each word of the subset of words for presentation to a subject in a speech test for the subject such that audio signals presented to the subject during the speech test are perceptually balanced.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a user wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a flowchart illustrating specific example embodiment of a process flow for generating a dynamic word list for use in a speech test;

FIG. 3 is a flowchart illustrating a generalized process flow for generating a dynamic word list for use in a speech test;

FIG. 4 is a first user interface that includes dynamically generated possible answers presented to a subject during a speech test;

FIG. 5 is a second user interface that includes dynamically generated possible answers presented to a subject during a speech test;

FIG. 6 is a flowchart illustrating a generalized process for conducting a speech test for a subject;

FIG. 7 illustrates a cochlear implant fitting system with which aspects of the techniques presented herein can be implemented; and

FIG. 8 is a schematic diagram illustrating an implantable stimulation system to which aspects of the techniques presented herein can be applied.

### DETAILED DESCRIPTION

Presented herein are techniques for dynamically generating test materials for administration of speech tests to, for example, hearing device users (e.g., users of hearing aids, cochlear implants, etc.). In particular, dynamic word list generation techniques are disclosed in which word lists are generated with specific attributes that are optimally suited to implement specific speech tests. For example, the disclosed techniques may be used to generate word lists with predetermined phonetic balance and/or perceptual balance. The disclosed techniques also provide for the generation of audio signals associated with the word lists used during the speech tests. For example, audio signals with specific levels of volume, signal-to-noise ratios, or speaker characteristics (e.g., identity, gender, pitch, timbre, speed of pronunciation, etc.) may be generated. Finally, the disclosed techniques also provide for dynamically generated user interfaces to implement speech tests.

Merely for ease of description, the techniques presented herein are primarily described with reference to a specific implantable medical device system, namely a cochlear implant system. However, it is to be appreciated that the techniques presented herein may also be partially or fully implemented by other types of implantable medical devices. For example, the techniques presented herein may be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein may also be implemented by dedicated tinnitus therapy devices and tinnitus therapy device systems. In further embodiments, the presented herein may also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

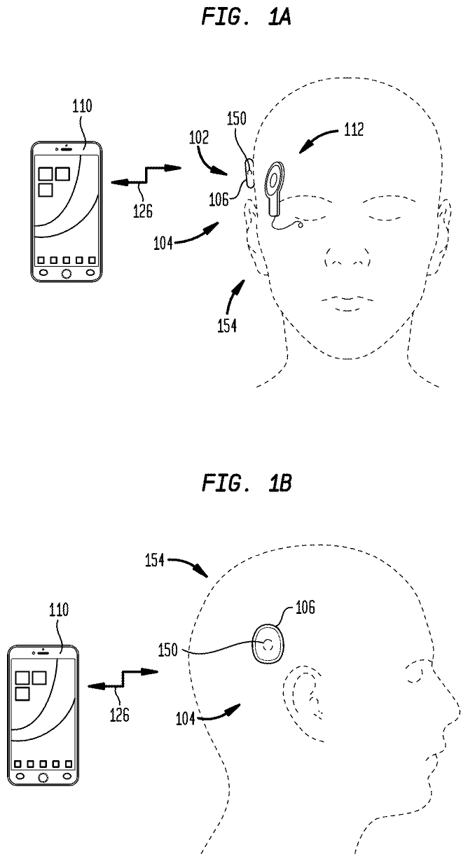

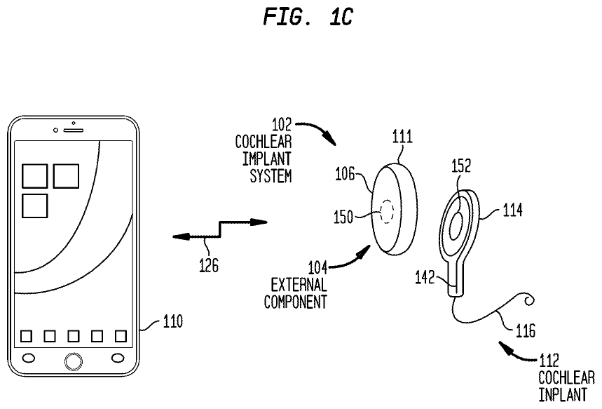

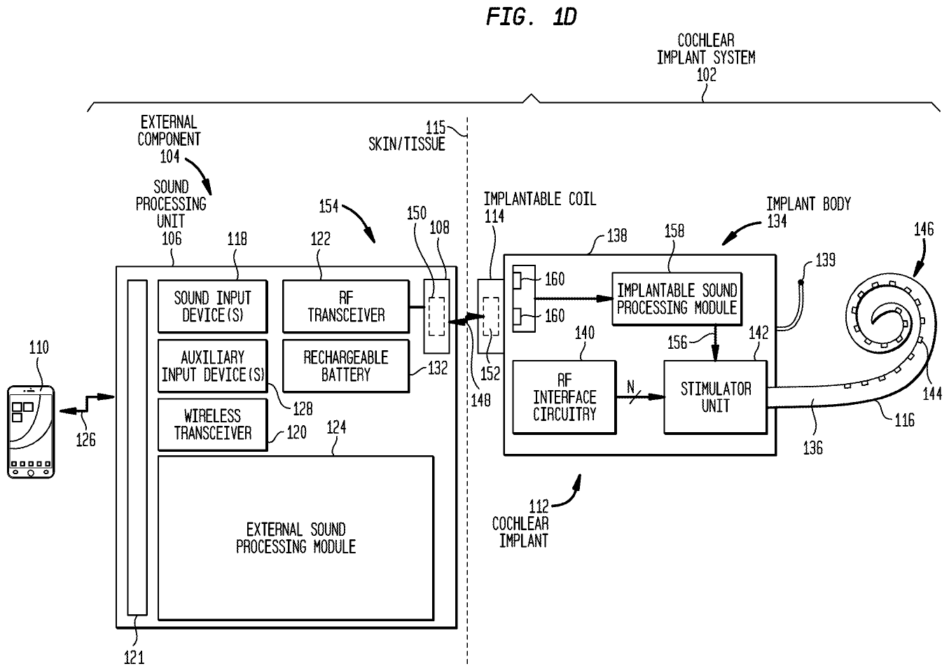

FIGS. 1A-1D illustrates an example cochlear implant system 102 with which aspects of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 154 of a user, while FIG. 1B is a schematic drawing of the external component 104 worn on the head 154 of the user. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the user and an implantable component 112 configured to be implanted in the user. In the examples of FIGS. 1A-ID, the external component 104 comprises a sound processing unit 106, while the cochlear implant 112 includes an implantable coil 114, an implant body 134, and an elongate stimulating assembly 116 configured to be implanted in the user's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the user's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component may comprise a behind-the-ear (BTE) sound processing unit or a micro-BTE sound processing unit and a separate external. In general, a BTE sound processing unit comprises a housing that is shaped to be worn on the outer ear of the user and is connected to the separate external coil assembly via a cable, where the external coil assembly is configured to be magnetically and inductively coupled to the implantable coil 114. It is also to be appreciated that alternative external components could be located in the user's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the user. For example, the cochlear implant 112 can operate in a first general mode, sometimes referred to as an “external hearing mode,” in which the sound processing unit 106 captures sound signals which are then used as the basis for delivering stimulation signals to the user. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the user. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110 is a computing device, such as a computer (e.g., laptop, desktop, tablet), a mobile phone, remote control unit, etc. As described further below, the external device 110 comprises a speech test module that, as described further below, is configured to implement aspects of the speech test techniques presented herein. According to the techniques presented herein, external device 110 may be used to provide a speech test to the user. Specifically, a speaker associated with external device 110 may be used to play audio signals associated with a list of words dynamically generated to meet the intended goals of the speech test. The display of external device 110 displays possible answers of words that may correspond to the word contained in the audio signal. The user then selects the appropriate one of the possible answers through user input to external device 110. The external device 110 and the cochlear implant system 102 (e.g., OTE sound processing unit 106 or the cochlear implant 112) wirelessly communicate via a bi-directional communication link 126. The bi-directional communication link 126 may comprise, for example, a short-range communication, such as Bluetooth link, Bluetooth Low Energy (BLE) link, a proprietary link, etc.

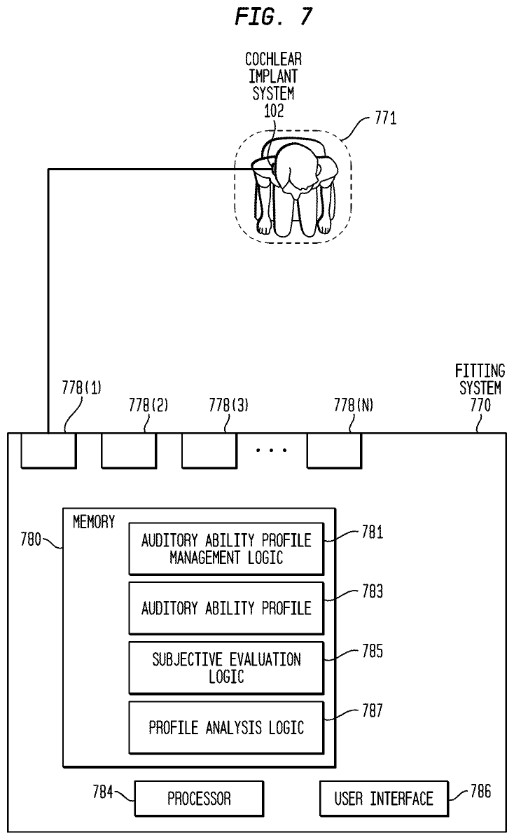

External device 110 may also be configured to communicate externally via, for example, a connection to the internet. Such a connection allows external device 110 to communicate the results of the speech tests to external entities, such as clinicians and/or audio prosthesis providers. These entities may use the results of the speech tests to tailor therapy to the specific user, better fit cochlear implant system 102 to the user (via a fitting system as described with reference to FIG. 7 below) and serve as the basis for additional research and improvements to audio prosthesis technologies, among other uses.

Returning to the example of FIGS. 1A-1D, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices may include additional types of input devices and/or less input devices (e.g., the wireless short range radio transceiver 120 and/or one or more auxiliary input devices 128 could be omitted).

The OTE sound processing unit 106 also comprises the external coil 108, a charging coil 121, a closely-coupled transmitter/receiver (RF transceiver) 122, sometimes referred to as or radio-frequency (RF) transceiver 122, at least one rechargeable battery 132, and an external sound processing module 124. The external sound processing module 124 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the user. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the user's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the user's cochlea.

Stimulating assembly 116 extends through an opening in the user's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a user (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the user.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the user's cochlea. In this way, cochlear implant system 102 electrically stimulates the user's auditory nerve cells, bypassing absent or defective hair cells that normally transduce acoustic vibrations into neural activity, in a manner that causes the user to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the user's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a user (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the user's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the user.

A typical hearing device, such as a cochlear implant, has a number of settings or parameters that determine the sound processing operations of the device. The individualized programs, commands, data, settings, parameters, instructions, modes, and/or other information that define the specific characteristics used by a hearing device, such cochlear implant system 102, to process electrical input signals and generate stimulation data therefrom are generally and collectively referred to as “sound processing settings.” These parameters are determined and set by medical practitioners (clinicians) in a process known as “fitting.” As described in detail below with reference to FIG. 7, external device 110 may be configured to send data to a fitting system (see fitting system 770 of FIG. 7) via, for example, a computer network, such as a wide area network (WAN) or a local area network (LAN). This permits clinicians to use online technologies to individualize the programs, commands, data, settings, parameters, instructions, modes, and/or other information that define the specific characteristics used by cochlear implant system 102.

Determining the parameters set during a fitting process may be based upon the results of specific testing of users. These tests may be used to determine how well users perform with their hearing implants. Unfortunately, relying on related art techniques for providing such tests often provide only limited insight into how well users perform with their medical device. For example, the related art techniques used to measure hearing outcomes may be inconvenient without integration into the medical device platforms, resulting in tests and test data that can be inaccessible for further data analysis.

A more beneficial approach may be to provide one or more performance metrics that capture the essence of the therapy outcomes that the medical device delivers. Unfortunately, in the field of hearing devices, such uniform measures may not be widely available. The closest proxy for such universal measures may be speech audiometry. Speech audiometry refers to a language dependent test. The core test in speech audiometry presents lists of words in the absence of background noise, and measures the basic speech understanding of the words by the test subject. Speech audiometry is used for characterization of the degree and the nature of a hearing loss in a test subject and for validation of treatment provided by medical devices, such as cochlear implant devices, hearing aids, and other solutions used to restore hearing.

Depending on the exact use case, the test can be taken in many variations:
Tests may vary by the word lists used. For example, the word lists used to test adults may be different than the word lists used to test children.
Test may vary by language, not only in the specific words used to test the subjects, but also in the phonemes presented to the subjects.
Tests may vary in the amount of background noise accompanying the test audio.
Tests may test one or both ears of the subject.
Tests may present the test audio through bone conduction, air conduction or both.
Test audio may be presented to the subject via headphones, with or without the subject's hearing device.
Tests may be presented with different signal to noise ratios (SNR).
Tests may test for a fixed speech recognition threshold (SRT) or the test may adapt to probe the limits of the subject's SRT.
Tests may be scored using different scoring techniques, such as word scoring or phoneme scoring techniques.

There are several major hurdles currently preventing wide adoption of a short words hearing test as a standard metric for hearing therapies. These hurdles may include:
1. Speech audiometry tests require expensive equipment, such as sound booths and digital audiometers, that generally are only available in specialized centers.
2. Classic speech audiometry requires a specialized professional (i.e., an audiologist) trained in proctoring speech audiometry tests. Moreover, the subject experience is poor. Taking a test is stressful and unpleasant: it feels like a school examination that the subject might fail.
3. Speech audiometry requires specialized speech materials. For clinical diagnostic purposes official speech lists have been carefully developed according to international standards, such as the International Organization for Standardization (ISO) standard 8253. Meeting these standards may involve validation studies to establish the diagnostic validity of the specific test materials. Only a few official speech audiometry lists exist in any given language. It may be detrimental to over-use these materials outside of a diagnostic setting as there is a risk of subjects learning the content of the lists, biasing future hearing assessments. Therefore, these official speech materials should be reserved for clinical evaluation of subjects.

Accordingly, clinical speech audiometry tests may be expensive, and their use of expensive and specialized equipment may be problematic. For example, in clinical speech audiometry, an audiologist is controlling an expensive audiometer, delivering the official speech audiometry materials through calibrated receivers in a shielded sound booth in a specialized audiological center. Access to such equipment may require substantial travel to a clinic, and be limited by the availability of a professional. Due to the limited availability of such speech audiometry tests, and because the results of such tests are held in the medical record systems of speech audiometry professionals, the results of such tests may not be available to those who develop and fit hearing devices.

Additionally, while the above-described clinical setup fulfills an important need when high precision is required for impactful professional medical decision making, this is not always a strict necessity. For example, the speech audiometry test results used by a cochlear implant automatic fitting systems only need to be sufficiently reliable to correlate well with in-clinic testing. Moreover, classic speech audiometry is not a precision metric with respect to systematic errors made by specific users of hearing devices. Classic speech audiometry provides an average word score useful for clinical verification and validation, but it does not provide detailed and precise information of what specific errors have been made by a particular subject. Therefore, classic speech audiometry may not always be particularly useful to drive further device fitting changes or to drive a next set of hearing exercises in a personalized hearing training program. For those purposes, the lists would have to be longer, which is not possible in a clinical setting.

Furthermore, the experience during traditional speech audiometry can be stressful for the subject. For example, some subjects may approach the speech audiometry in an adversarial way, like an examination instead of as an evaluation, which can impact the results of the evaluation. In speech audiometry the hearing tests are typically difficult, exploring the boundaries of hearing of the test subject. The subjects may struggle at these boundaries, and because the subjects are encouraged to keep reporting whatever speech fragments they have understood, subjects may guess or gamble at the correct response. Furthermore, personality may influence the evaluations, particularly for subjects predisposed to feel embarrassed by how little they may understand. These types of feelings and anxieties may result in subjects holding back from expressing what they believe they understood.

An additional challenge presented by speech audiometry is that, in every language, the official clinical materials are unique and very valuable. The official speech audiometry batteries have been developed with great care, selecting words that are ideally phonetically and perceptually balanced. Both phonetic and perceptual balance may be required for valid speech audiometry materials, at least according to ISO 8253. Phonetical balance refers to the phoneme distribution of the word list. A phonetically balanced word list in the corpus is on average identical and corresponds to the statistics of the language. Perceptual balance refers to the equivalency of the list to other lists regarding how test participants are able to perceive the test materials. It should not matter whether list A or list B is used to test the speech understanding of a subject. Perceptual balance ensures that a test gives the same hearing estimate regardless of the word list used. Meeting these phonetic and perceptual balance goals may require a painstakingly slow process of carefully selecting and composing speech materials and running multiple validation studies with normal hearing young adults. It is therefore problematic to re-use the official speech audiometry materials for home testing. Typically, there are only few lists with not many words (e.g. in Dutch 15 lists containing 11 words). Offering them repeatedly in a home test would create a risk of learning these lists by heart, causing bias on the official speech audiometry tests in the clinic. For home testing, the ideal test can be repeated infinitely without the risk of a learning effect due to memorizing the word lists.

Overcoming these challenges may provide substantial benefits for hearing device users. For example, overcoming these challenges may allow for at-home speech audiometry that allows subjects (e.g., users of hearing devices, such as cochlear implant users) to perform speech tests in their home environment. Furthermore, the results of at-home tests may have validity for a number of relevant clinical questions, such as tracking performance. For example, the test results may reflect how a person is doing in terms of their hearing performance, without necessarily being exactly equivalent to the high standards required in an in-clinic test. Most adult users will be eager to improve their hearing with their new device, certainly in the first weeks and months after implantation. They are highly motivated to reach their hearing potential and may appreciate the ability to conduct hearing games and hearing tests.

Furthermore, readily available home speech audiometry may facilitate substantial innovation. For example, if more robust data sets are more readily available, it may be possible to innovate in the field of hearing devices and hearing devices fitting systems. For example, more robust and available speech audiometry data sets may facilitate the development of closed loop AI fitting systems based on speech audiometry outcomes. Other possible innovations may include the development of unbiased and trustworthy outcome prediction models.

In order to provide for robust home speech audiometry, the techniques of the present disclosure enable development of specialized language materials (e.g., phonetically and perceptually balanced words lists), other than the in-clinic speech audiometry materials. In other words, the techniques of this disclosure provide for speech audiometry materials that can be repeatedly performed outside of a clinical environment, such as in the subject's home via a computing device, such as external device 110 of FIGS. 1A-1D. Accordingly, the techniques of the present disclosure may provide one or more of the following benefits:
Dynamic speech lists that allow for repeated speech audiometry testing without risk of learning effects;
Speech lists that are phonemically balanced;
Speech lists that are perceptually balanced;
Low development effort per language to develop test materials;
Speech lists composed of a finite test corpus, containing no more than +/−1000 short words;
Elimination of the need for a grapheme-to-phoneme (g2p) component for every language. This component requires considerable work to transcribe open set answers or word fragments and is not readily available on the open market. Depending on the spelling difficulty for a particular language, it can be a significant hurdle and cost;
The elimination of extensive validation studies; and
Immediate feedback that can be used to motivate subjects to continue testing and rehabilitation procedures.

According to one aspect of the techniques presented herein is the generation of “dynamic” lists of speech test materials. Dynamic lists are randomly generated by a processing device, such as a computer like external device 110 of FIGS. 1A-1D. The words in the dynamic lists are selected from a word pool to have properties that meet the goals of certain tests, such as phonetic and/or perceptual balance. These dynamic lists are then used to perform, for example, in-home speech audiometry examination. The use of such in-home tests may provide a precise screener for inadequate performance by hearing prosthetics, or for characterization of the degree of hearing loss in general. Furthermore, because the tests are implemented via applications that run on subjects' computing devices (e.g., external device 110 of FIGS. 1A-1D), the tests may be performed on a regular basis. Additionally, the tests may be incorporated into games or provide positive feedback with respect to subject progress to help motive subjects to perform the tests. For example, the application may set goals or provide rewards to subjects in response to performing specific tests.

Finally, allowing subjects to perform in-home tests will greatly increase the amount of data available to evaluate error patterns specific to a particular subject. For example, it is suspected that subjects make systematic mistakes in terms of the phonemes they misrecognize that may not be readily apparent from-in-clinic speech tests. Accordingly, two subjects that score an identical 60% on in-clinic speech audiometry tests may actually be making very different mistakes, and the differences in the mistakes may not be readily apparent from the few in-clinic mistakes that a subject normally undertakes. Knowing the differences in these mistakes may allow the two subjects to have their hearing devices more appropriately fitted to their respective hearing deficiencies. Currently, there is simply not enough readily available data to determine how two subjects with the same scores on their speech audiometry tests systematically differ. Having subjects regularly perform speech audiometry tests in their home environment may generate a sufficient volume of data that provides greater precision in estimating the personalized error patterns of specific subjects. Once specific error patterns are recognized, subjects may be provided with optimized audio maps or offered specialized rehabilitation training to help the subjects discriminate similar sounds.

The first step in generating the dynamic word lists of the techniques disclosed herein is determining the composition of a word pool. The contents of the word pool should all have a very similar phonetic structure, e.g. a consonant-vowel-consonant (CVC) structure, and be well known words of the language of the test such that they are highly familiar to most subjects. Audio recordings of high quality natural samples of the words in the word pool should be provided, such as high quality recordings, uttered by a professional speaker using a standard pronunciation for each word. According to other embodiments, speech synthesis technologies (e.g., text-to-speech technologies) may be used to generate high-quality computer voice audio data for the words in the word pool. The use of speech synthesis technologies may decrease costs and the time needed to acquire the high quality audio data. The use of speech synthesis technologies may have added benefits, such as standardizing the speaker characteristics (identity, gender, pitch, timbre, speed of pronunciation, and others known to the skilled artisan) across languages. Such standardization may result in more uniform scores across languages.

Once the word pool is populated, an algorithm may be used generate the word lists as subsets of the word pool. If a phonetically balanced word lists is needed for a particular test, the algorithm tracks the average phoneme distribution of the word pool. When selecting words to compose a new list, the algorithms strives to match this phoneme distribution. For example, the algorithm may randomly select a first word for a list. For subsequent words, the algorithm may search the word pool in a random fashion for different words closing the phoneme distribution gap. According to specific embodiments, the algorithm selects a random word to add to the list. If the selected word brings the phoneme distribution closer to the known average phoneme distribution of the world pool, the selected word is added to the word list. On the other hand, if the selected word drives the phoneme distribution of the list away from the average phoneme distribution of the word pool, the word is discarded. This process is repeated until the word list is generated with, for example, a predetermined number of words and an average phoneme distribution within a threshold of the average phoneme distribution of the word list.

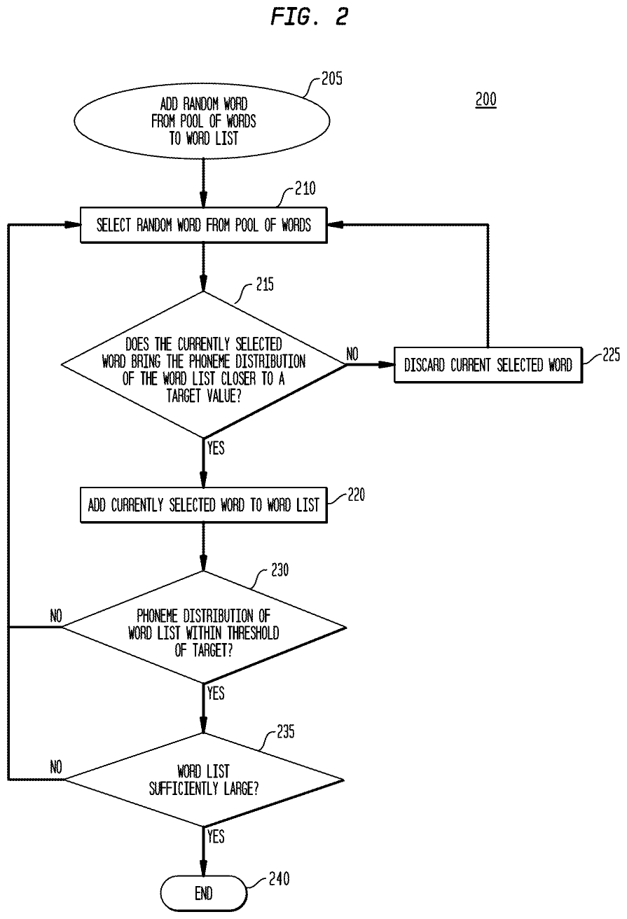

With reference now made to FIG. 2, depicted therein is a flowchart 200 illustrating an example search algorithm configured to implement the dynamic speech audiometry test word list generation techniques of the present disclosure. Flowchart 200 begins in operation 205 where a random word is selected from a pool of words and added to a word list. In operation 210, another word is randomly selected from the pool of words. If it is determined in operation 215 that the currently selected word brings the phoneme distribution of the word list closer to a target value, the word is added to the word list in operation 220. On the other hand, if it is determined that the currently selected word drives the phoneme distribution of the word list farther from the target value, the word is discarded in operation 225, and the process flow returns to operation 210.

The target phoneme distribution discussed in operation 215 may be the average phoneme distribution of the pool of words, or it may be another phoneme distribution. For example, the target phone distribution may be selected so that the word list generated by flowchart 200 is configured to test a particular feature of a subject's hearing.

Returning to operation 220, after the currently selected word is added to the word list, it is determined in operation 230 whether or not the phoneme distribution is within a threshold of the target value. If the phoneme distribution is not yet within the threshold of the target value, the processing of flowchart 200 returns to operation 210 and another word is randomly selected from the pool of words.

On the other hand, if the phoneme distribution is within the threshold of the target value, the process flow of flowchart 200 proceeds to operation 235. Operation 235 determines whether or not the word list is sufficiently large to implement the intended speech audiometry test. If the word list is not yet sufficiently large, the processing of flowchart returns to operation 210 and another word is randomly selected from the pool of words. If the word list is sufficiently large, the processing of flowchart 200 proceeds to operation 240, and the search algorithm is complete.

Accordingly, flowchart 200 provides a process flow in which operations 210-235 are repeated until a word list is generated of sufficient size with a phoneme distribution within a threshold value of a target value. The word list may them be used to implement a speech audiometry test.

Flowchart 200 is just one example of how the words selected for inclusion in a test word list may be determined. According to another example, after the first word is randomly selected for inclusion in the test word list, the remaining words in the pool of words may be scored to determine how much each word will drive the phoneme distribution of the test word list towards the target phoneme distribution. A next word for inclusion the test word list may be randomly selected from the words with the best scores. This process would then repeat until the test word list is populated with a phoneme distribution within a threshold value of the target phoneme distribution and with a sufficient number of words. For example, after selection of the second word, the remaining words in the pool of words would be scored again, but this time based upon the phoneme distribution provided by the first and second words. A third word would then be randomly selected from the words with the best scores. This process would then be repeated as necessary to populate a test word list of sufficient size.

The above described search algorithms, and other algorithms known to the skilled artisan, may be embodied as an artificial intelligence (AI) element, such as a machine learning algorithm or model, that is based on phoneme allocation tables. The use of such an AI element may guarantee that the dynamic word lists are phonemically balanced.

In addition to the phonemically balanced word selection techniques described above, the techniques of the present disclosure also provide for generating perceptually balanced word lists. The perceptual balance of a word list may be a function of the specific words in the list, as well as the quality of the audio recordings associated with the words. For example, certain common words may be easily perceived based on their prevalence within a specific language. Perceptual balance may also be affected by the volume of the audio signal associated with word and the SNR in the audio signal associated with the word. Accordingly, when dynamically generating a word list for a speech audiometry test, the perceptual balance of the word list may also be a consideration. For example, when selecting words from the pool of words for inclusion in the word list, the prevalence of the word within the language may be a criteria considered when determining whether or not to include a word in the word list. Similarly, the qualities of the audio recordings associated with the word may also be considered. For example, when selecting words for inclusion in a dynamic word list, the SNR of the audio recording associated with the word may be evaluated to ensure perceptual balance of the word list. Similarly, when a word is selected for inclusion in the dynamic word list, a volume setting for the audio signal associated with the word may also be set to ensure perceptual balance of the word list. The techniques of the present disclosure may leverage computer models configured to predict the intelligibility of an utterance. Such computer models may assign utterances a speech intelligence index or speech articulation index. Based upon such index values, the perceptual balance of a test word list may be determined such that the final test word list has a perceptual balance within a predetermined threshold of a target perceptual balance value.

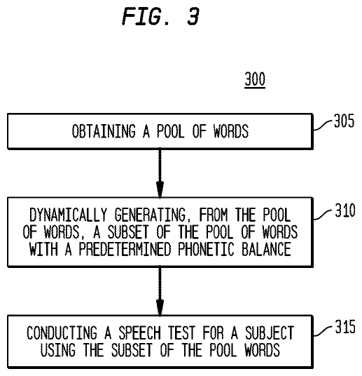

With reference now made to FIG. 3, depicted therein is a flowchart 300 illustrating a generalized process flow for implementing the dynamic word list generation techniques of the present disclosure.

Flowchart 300 begins in operation 305 in which a pool of words is obtained (e.g., generated, compiled, received, etc.). For example, operation 305 may include compiling a pool of words with a predetermined phoneme distribution. The pool of words may also be compiled such that a predetermined number of words within the pool of words have a similar phonetic structure, such as a CVC structure.

In operation 310, a subset of the pool of words is dynamically generated from the pool of words with a predetermined phonetic balance. For example, operation 310 may be embodied as the use of a search algorithm against the pool of words, such as the algorithm illustrated in flowchart 200 of FIG. 2. Because operation 310 may be embodied as a computer executed method, operation 310 may include the operations of applying a search algorithm to data indicative of the pool of words to generate data indicative of the subset of the pool of words such that a phonetic distribute of the subset of the pool of words is within a threshold of the predetermined phonetic distribution, followed by generating, from the data indicative of the subset of the pool of words, a test set of words for a speech test for a subject.

Finally, in operation 315, a speech test is conducted using the subset of the pool of words. For example, a speech audiometry examination may be performed using the subset of the pool of words. Operation 315 may include generating an audio signal for each word of the subset of words for presentation to a subject in the speech test. The audio signals presented to the subject during the speech test may be perceptually balanced. For example, the audio signals presented to the subject may be presented at different volume levels so that the overall test is perceptually balanced. The audio signals presented to the subject may also be presented with different levels of background noise or with different SNRs to ensure a perceptually balanced test. The volume, background noise and/or SNR levels may be a pre-existing feature of the audio signals presented during the speech test, or these features may be dynamically set during the speech test. Similarly, the audio signals themselves may be pre-existing, such as pre-existing audio recordings or pre-existing audio data files generated using speech synthesizing technologies. Alternatively, the audio signals may be dynamically generated using speech synthesizing technologies during the presentation of the speech test.

As noted through operation 315 of flowchart 300 of FIG. 3, the techniques of this disclosure include conducting speech tests using dynamically generated lists of words. The techniques of the present disclosure may implement such speech tests utilizing dynamically generated test answers based on the subset of the pool of words. A user interface may then be generated to implement the speech test by presenting the test answers to the subject as part of the speech test.

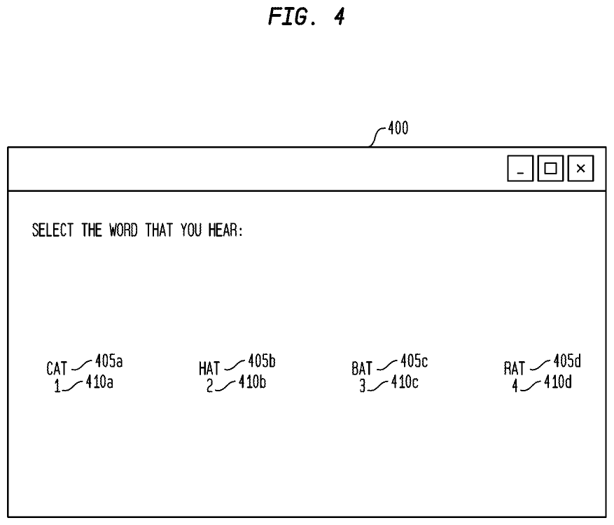

With reference now made to FIG. 4, depicted therein is a user interface 400 that may be dynamically generated to conduct a speech test according to the techniques of the present disclosure. User interface 400 may be implemented on an application executing on a computing device associated with the subject, such as the external device 110 of FIGS. 1A-ID. The application may present the audio signals to the subject via a speaker connected to the computing device (e.g., an internal speaker of the computing device, wired or wireless headphones connected to the computing device, etc.) while simultaneously presenting the subject with user interface 400 via a display of the computing device.

User interface 400 includes a limited number of possible answers 405a-d that a subject may indicate as corresponding to the audio signal played as part of the speech test. Accordingly, the subject will hear an audio signal of one of the words from the dynamically generated word list. The subject will then select the one of possible answers 405a-d that the subject believes corresponds to the audio signal. Each of possible answers 405a-d includes an associated response value 410a-d, respectively, which a subject may input via a user input device, examples of which include keyboards and number pads. While four possible answers 405a-d are displayed in user interface 400, other embodiments may provide the subject with more or fewer responses. For example, a traditional number pad has values “0” through “9,” providing inputs for up to ten possible answers. According to other examples, the subject may select one of the possible answers 405a-d using a computer mouse, stylus or touchscreen functionality of the screen on which possible answers 405a-d are displayed. Once the subject has input an answer, user interface 400 may update with a new set of possible answers 405a-d, and the subject will be presented with a new audio signal corresponding to another one of the words from the dynamically generated word list.

User interface 400 may differ from how related art speech audiometry tests are performed in clinical settings. In clinical environments, “open set recognition” may be used. Specifically, in clinical speech audiometry tests, the subject is not presented with possible answers. Instead, the subject may be provided with a keyboard via which they may type out any word that they believe they heard as part of the test. Because the subject can enter any word, the set of possible answers is “open.” User interface 400, on the other hand, provides a limited number of possibilities to the subject. The subject selects what it believes to be the correct word from the possible answers 405a-d. Accordingly, where clinical speech audiometry test may use open set recognition, user interface 400 provides a closed set of options.

Nevertheless, the closed set techniques of the present disclosure may be implemented so that the results of the tests are substantively equivalent to open set techniques. For example, if nine possible answer options are presented to the subject, the probability that the subject would randomly select the correct answer is only 11%. This low probability may be sufficient to reach near-equivalence with open set recognition techniques. Furthermore, the number of possible answers 405a-d may be selected in order to provide a speech audiometry test that is equivalent to an open set test within a predetermined error threshold.

The techniques of the present disclosure also include techniques directed to determining which values should be presented to the subject as possible answers 405a-d. The possible answers 405a-d may be selected such that they dynamically adapt the confounders to the word being presented to the subject via the audio signal. For example, if the speech audiometry test being presented is intended to be relatively easy, possible answers 405a-d may be selected such that the incorrect answers are phonemically very distinct from the correct answer. If the intent is to make the test more challenging, possible answers 405a-d may be selected such that the incorrect answers differ from the correct answers in only a single phoneme. In other words, possible answers 405a-d may be selected according to their average phonetic distance from the word from the correct answer. Accordingly, possible answers 405a-d may be selected such that the test results using the closed set techniques of user interface 400 achieve equivalent results to the related art in-clinic open-set tests.

Other embodiments may intend to optimize the incorrect possible answers towards estimating phoneme discrimination by the subject, estimating a phoneme confusion matrix, and/or identifying the phoneme pairs that are most often confused by the subject. Accordingly, possible answers 405a-d may be selected such that the results of the speech audiometry test provide a reliable estimate of the subject's discrimination capability at the phoneme level.

The closed set techniques of user interface 400 may also eliminate the need for a g2p component to the test. Because the subject can only select from the options shown on the screen, all possible responses are known beforehand. The responses may belong to a second response word pool, which may be a super set of the word pool from which the dynamic word list was generated. Accordingly, only substitution errors may be possible. The techniques of the present disclosure also allow for the inclusion of an “I Do Not Know” or “None of the Above” option in possible answers 405a-d. By including these options, a speech audiometry test provided by user interface 400 may test for whole word deletion errors.

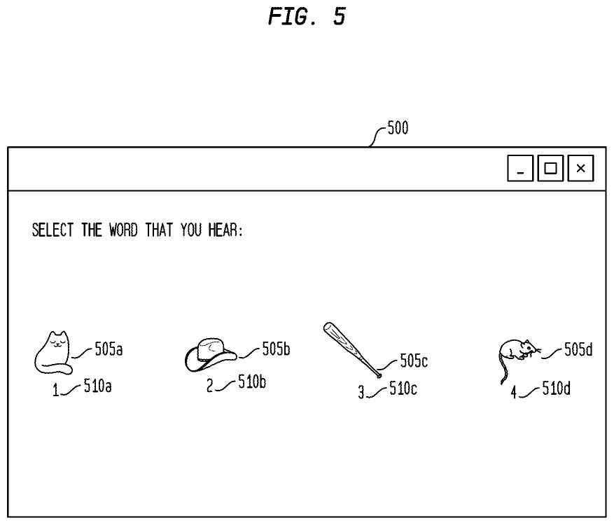

The techniques of the present disclosure may also be adapted to facilitate speech audiometry tests for children or those with reading difficulties, as illustrated in user interface 500 of FIG. 5. User interface 500 is similar to user interface 400 of FIG. 4, but differs in that possible answers 505a-d are presented as images. Accordingly, user interface 500 may be used to provide speech audiometry tests to subjects with difficulty reading and/or subjects who have not yet learned to read. In other words, subjects who would be unable to read possible answers 405a-d of FIG. 4 can identify the images of possible answers 505a-d, allowing the subject to select the correct associated response value 510a-d.

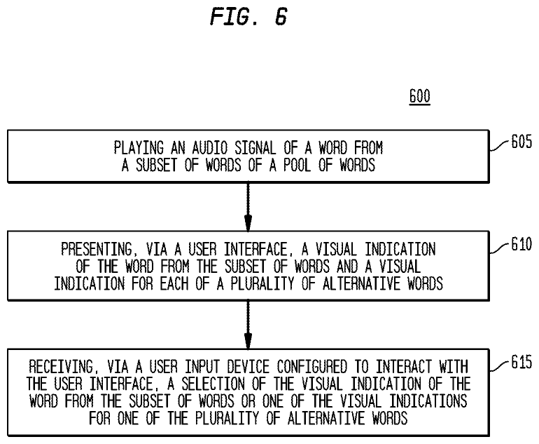

Turning now to FIG. 6, depicted therein is a flowchart 600 that provides a process flow for implementing the speech audiometry test techniques of the present disclosure. Flowchart 600 begins in operation 605 in which an audio signal of a word of a subset of a pool of words is played for a subject. Accordingly, operation 605 may be embodied as the playing of an audio recording of one of the words in a dynamically generated list of words, such as a list of words generated as described above with reference to FIGS. 2 and 3. Operation 605 may also be embodied as the generation of an audio signal via speech synthetization technologies, and then presenting the audio signal to the subject. The presentation of the audio signal to the subject in operation 605 may be done so that the speech test is perceptually balanced. Accordingly, operation 605 may include presenting audio signals with different volumes, different levels of background noise, and/or different SNRs.

In operation 610, a visual indication of the word is presented to the subject via a user interface, as are visual indications for each of a plurality of alternative words. Accordingly, operation 610 may be embodied as the display or presentation of a user interface analogous to user interface 400 of FIG. 4 or user interface 500 of FIG. 5. As discussed above, the visual indications for the alternative words may be selected to, for example, provided a test with a predetermined level of difficulty, provide a test that evaluates a discrimination loss for the subject, provide a test that determines a sensitivity loss for the subject, or otherwise tailor the test as understood by the skilled artisan.

In operation 615, a selection of the visual indication of the word from the subset of words or one of the visual indications for one of the plurality of alternative words is received. The input is received via a user input device configured to interact with the user interface.

Once a test is completed via the process flow of flowchart 600, the results of the test may be scored. For example, phonetic transcription of the target words and subject responses are compared. The most dominant phoneme errors may be provided to an audio professional or the phoneme confusion matrix for the subject may be calculated. Average reaction time between the playing of the audio signal in operation 605 and the receipt of a response in operation 615 may also be used in the scoring. The measure of such reaction times may indicate the listening effort of the subject during the test. Additional scoring techniques may also be used without deviating from the techniques disclosed herein. For example, the techniques disclosed herein may leverage the scoring techniques disclosed in International Application No. PCT/IB2020/000952 (designating the United States and published under International Publication No. WO/2021/099834 on May 27, 2021), the contents of which are hereby incorporated by reference in their entirety. Once scored, the results of the test may then be used to tailor a subject's treatment, such as tailoring the fitting of a hearing device as described below with reference to FIG. 7.

Through the above-described techniques, test materials specific hearing tests (e.g., word lists and audio signals or recorders) may be provided with decreased effort compared to related art techniques. Furthermore, the techniques of the present disclosure allow for additional benefits and provide for additional use cases, including building suites of hearing tests of varying difficulty. Such suites of tests may then be used to implement hearing games for supporting rehabilitation. The suites of test can provide the following different variations:
Tests that vary in presentation level, i.e. tests that are soft versus tests that are loud.
Tests with varying word lists. Some lists may be phonemically balanced, while other may be configured to target or train specific phonemes.
Tests can be generated that add background noise to the test audio signals to assist in the training of hearing in noisy environments. Such tests may have a fixed SNR or the SNR in such tests may vary.
Adaptive tests. Because the techniques provided herein dynamically generate word lists and audio signals used to run the tests, test materials can be generated that search for a subject's speech recognition threshold SRT. SRT may be tested based upon varying presentation level (i.e., volume), varying SRT, or both. The techniques presented herein may vary these values “on the fly” during a speech test.
Tests can be generated to train working memory by offering word triplets instead of single words. For example, a subject may be played audio signals associated with three, and asked to identify the words only after the audio for all three has completed. Subjects may be tasked with entering answer for the words in forward or reverse order. The test may also measure auditory memory by lengthening the word sequence to 4, 5, 6 or more words to be entered in forward or reverse order.
The tests may evaluate listening effort by measuring reaction time or by increasing the number of words used to test listening fatigue.
Tests may be generated that force subjects to respond within a certain time to train their reaction speed.
Tests can be generated that train subjects to recognize different types of speech. For example, the use of text-to-speech technologies may allow for audio signals associated with the dynamically generated word lists to be generated using a mix of different voices, including a mix of male voices, female voices, voices with regional pronunciations, and voices that speak at different rates.
Tests may be generated to test or train subjects to recognize speech with varying acoustics. For example, reverberation or telephone filtering may be added to the audio signals presented to the subject during the tests.
The tests may be proctored with immediate feedback to the subject. For example, after a subject enters their answer, the user interface may highlight the correct answer to expedite training.
Tests may be generated that measure tone recognition in tonal languages by offering tonal variants in the response set.

All of the-above described test types may then be used to tailor treatment or audio prosthesis fitting. Turning to FIG. 7, depicted therein is a block diagram illustrating an example fitting system 770 configured to fit a hearing device based, at least in part, on the speech tests performed according to the techniques presented herein. Fitting system 770 is, in general, a computing device that comprises a plurality of interfaces/ports 778(1)-778(N), a memory 780, a processor 784, and a user interface 786. The interfaces 778(1)-778(N) may comprise, for example, any combination of network ports (e.g., Ethernet ports), wireless network interfaces, Universal Serial Bus (USB) ports, Institute of Electrical and Electronics Engineers (IEEE) 1394 interfaces, PS/2 ports, etc. In the example of FIG. 7, interface 778(1) is connected to cochlear implant system 102 having components implanted in a user 771. Interface 778(1) may be directly connected to the cochlear implant system 102 or connected to an external device that is communication with the cochlear implant systems (e.g., external device 110 of FIGS. 1A-1D). Interface 778(1) may be configured to communicate with cochlear implant system 102 via a wired or wireless connection (e.g., telemetry, Bluetooth, etc.).

The user interface 786 includes one or more output devices, such as a display screen (e.g., a liquid crystal display (LCD)) and a speaker, for presentation of visual or audible information to a clinician, audiologist, or other user. The user interface 786 may also comprise one or more input devices that include, for example, a keypad, keyboard, mouse, touchscreen, etc.

The memory 780 comprises auditory ability profile management logic 781 that may be executed to generate or update a user's auditory ability profile 783 that is stored in the memory 780. The auditory ability profile management logic 781 may be executed to obtain the results of objective evaluations of a user's cognitive auditory ability from an external device, such as an imaging system (not shown in FIG. 7), via one of the other interfaces 778(2)-778(N). The auditory ability profile management logic 781 may also be executed to obtain speech test data acquired using the techniques disclosed herein. For example, auditory ability profile management logic 781 may connect to the external device 110 of FIGS. 1A-1D using one of the other interfaces 778(2)-778(N). The auditory ability profile management logic may download speech test results data from the external device, and use the speech test results to generate or update a user's auditory ability profile 783. Alternatively, auditory ability profile management logic 781 may download speech test results from an external server or computing device via a network connection, such as the internet. This externally acquired data may then be used by auditory ability profile management logic 781 to generate or update a user's auditory ability profile 783.

In certain embodiments, memory 780 comprises subjective evaluation logic 785 that is configured to perform subjective evaluations of a user's cognitive auditory ability and provide the results for use by the auditory ability profile management logic 781. Subjective evaluation logic 785 may include the applications described above that are executed to provide the speech test techniques disclosed herein. Accordingly, fitting system 770 may not only use the results of speech tests to fit the cochlear implant system 102, fitting system 770 may also be used to perform the speech tests of the present disclosure. In other embodiments, the subjective evaluation logic 785 is omitted and the auditory ability profile management logic 781 is executed to obtain the results of subjective evaluations of a user's cognitive auditory ability from an external device (not shown in FIG. 7), via one of the other interfaces 778(2)-778(N).

The memory 780 further comprises profile analysis logic 787. The profile analysis logic 787 is executed to analyze the user's auditory profile (i.e., the correlated results of the objective and subjective evaluations) to identify correlated stimulation parameters that are optimized for the user's cognitive auditory ability.

Memory 780 may comprise read only memory (ROM), random access memory (RAM), magnetic disk storage media devices, optical storage media devices, flash memory devices, electrical, optical, or other physical/tangible memory storage devices. The processor 784 is, for example, a microprocessor or microcontroller that executes instructions for the auditory ability profile management logic 781, the subjective evaluation logic 785, and the profile analysis logic 787. Thus, in general, the memory 780 may comprise one or more tangible (non-transitory) computer readable storage media (e.g., a memory device) encoded with software comprising computer executable instructions and when the software is executed (by the processor 784) it is operable to perform the techniques described herein.

The correlated stimulation parameters identified through execution of the profile analysis logic 787 are sent to the cochlear implant system 102 for instantiation as the cochlear implant's current correlated stimulation parameters. However, in certain embodiments, the correlated stimulation parameters identified through execution of the profile analysis logic 787 are first displayed at the user interface 786 for further evaluation and/or adjustment by a user. As such, the user has the ability to refine the correlated stimulation parameters before the stimulation parameters are sent to the cochlear implant system 102.

The general operations for analysis of the user's auditory profile to identify correlated stimulation parameters that are optimized for the user's cognitive auditory ability have been described above. However, it is to be appreciated that the profile analysis logic 787 may operate in accordance with one or more selected guidelines set by a user via the user interface 786. For example, a user may configure the stimulation parameters that may be adjusted or set limits for how a stimulation parameter may be adjusted.

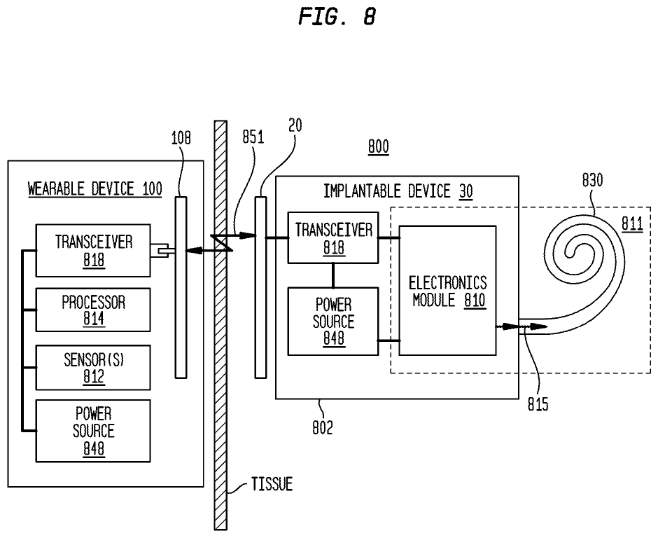

As previously described, the technology disclosed herein can be applied in any of a variety of circumstances and with a variety of different devices. Example devices that can benefit from technology disclosed herein are described in more detail in FIG. 8, below. As described below, the operating parameters for the devices described with reference to FIG. 8 may be configured using a fitting system analogous to fitting system 770 of FIG. 7. For example, the techniques described herein can be used to prioritize clinician tasks associated with configuring the operating parameters of wearable medical devices, such as an implantable stimulation system as described in FIG. 8. Further, technology described herein can also be applied to consumer devices. These different systems and devices can benefit from the technology described herein.

FIG. 8 is a functional block diagram of an implantable stimulator system 800 that can benefit from the technologies described herein. The implantable stimulator system 800 includes the wearable device 100 acting as an external processor device and an implantable device 30 acting as an implanted stimulator device. In examples, the implantable device 30 is an implantable stimulator device configured to be implanted beneath a user's tissue (e.g., skin). In examples, the implantable device 30 includes a biocompatible implantable housing 802. Here, the wearable device 100 is configured to transcutaneously couple with the implantable device 30 via a wireless connection to provide additional functionality to the implantable device 30.

In the illustrated example, the wearable device 100 includes one or more sensors 812, a processor 814, a transceiver 818, and a power source 848. The one or more sensors 812 can be one or more units configured to produce data based on sensed activities. In an example where the stimulation system 800 is an auditory prosthesis system, the one or more sensors 812 include sound input sensors, such as a microphone, an electrical input for an FM hearing system, other components for receiving sound input, or combinations thereof. Where the stimulation system 800 is a visual prosthesis system, the one or more sensors 812 can include one or more cameras or other visual sensors. Where the stimulation system 800 is a cardiac stimulator, the one or more sensors 812 can include cardiac monitors. The processor 814 can be a component (e.g., a central processing unit) configured to control stimulation provided by the implantable device 30. The stimulation can be controlled based on data from the sensor 812, a stimulation schedule, or other data. Where the stimulation system 800 is an auditory prosthesis, the processor 814 can be configured to convert sound signals received from the sensor(s) 812 (e.g., acting as a sound input unit) into signals 851. The transceiver 818 is configured to send the signals 851 in the form of power signals, data signals, combinations thereof (e.g., by interleaving the signals), or other signals. The transceiver 818 can also be configured to receive power or data. Stimulation signals can be generated by the processor 814 and transmitted, using the transceiver 818, to the implantable device 30 for use in providing stimulation.

In the illustrated example, the implantable device 30 includes a transceiver 818, a power source 848, and a medical instrument 811 that includes an electronics module 810 and a stimulator assembly 830. The implantable device 30 further includes a hermetically sealed, biocompatible implantable housing 802 enclosing one or more of the components.

The electronics module 810 can include one or more other components to provide medical device functionality. In many examples, the electronics module 810 includes one or more components for receiving a signal and converting the signal into the stimulation signal 815. The electronics module 810 can further include a stimulator unit. The electronics module 810 can generate or control delivery of the stimulation signals 815 to the stimulator assembly 830. In examples, the electronics module 810 includes one or more processors (e.g., central processing units or microcontrollers) coupled to memory components (e.g., flash memory) storing instructions that when executed cause performance of an operation. In examples, the electronics module 810 generates and monitors parameters associated with generating and delivering the stimulus (e.g., output voltage, output current, or line impedance). In examples, the electronics module 810 generates a telemetry signal (e.g., a data signal) that includes telemetry data. The electronics module 810 can send the telemetry signal to the wearable device 100 or store the telemetry signal in memory for later use or retrieval.

The stimulator assembly 830 can be a component configured to provide stimulation to target tissue. In the illustrated example, the stimulator assembly 830 is an electrode assembly that includes an array of electrode contacts disposed on a lead. The lead can be disposed proximate tissue to be stimulated. Where the system 800 is a cochlear implant system, the stimulator assembly 830 can be inserted into the user's cochlea. The stimulator assembly 830 can be configured to deliver stimulation signals 815 (e.g., electrical stimulation signals) generated by the electronics module 810 to the cochlea to cause the user to experience a hearing percept. In other examples, the stimulator assembly 830 is a vibratory actuator disposed inside or outside of a housing of the implantable device 30 and configured to generate vibrations. The vibratory actuator receives the stimulation signals 815 and, based thereon, generates a mechanical output force in the form of vibrations. The actuator can deliver the vibrations to the skull of the user in a manner that produces motion or vibration of the user's skull, thereby causing a hearing percept by activating the hair cells in the user's cochlea via cochlea fluid motion.

The transceivers 818 can be components configured to transcutaneously receive and/or transmit a signal 851 (e.g., a power signal and/or a data signal). The transceiver 818 can be a collection of one or more components that form part of a transcutaneous energy or data transfer system to transfer the signal 851 between the wearable device 100 and the implantable device 30. Various types of signal transfer, such as electromagnetic, capacitive, and inductive transfer, can be used to usably receive or transmit the signal 851. The transceiver 818 can include or be electrically connected to a coil 20.

As illustrated, the wearable device 100 includes a coil 108 for transcutaneous transfer of signals with the concave coil 20. As noted above, the transcutaneous transfer of signals between coil 108 and the coil 20 can include the transfer of power and/or data from the coil 108 to the coil 20 and/or the transfer of data from coil 20 to the coil 108. The power source 848 can be one or more components configured to provide operational power to other components. The power source 848 can be or include one or more rechargeable batteries. Power for the batteries can be received from a source and stored in the battery. The power can then be distributed to the other components as needed for operation.

As should be appreciated, while particular components are described in conjunction with FIG. 8, technology disclosed herein can be applied in any of a variety of circumstances. The above discussion is not meant to suggest that the disclosed techniques are only suitable for implementation within systems akin to that illustrated in and described with respect to FIG. 8. In general, additional configurations can be used to practice the methods and systems herein and/or some aspects described can be excluded without departing from the methods and systems disclosed herein.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A method comprising:
obtaining a pool of words;
dynamically generating, from the pool of words, a subset of the pool of words with a predetermined phonetic balance; and
conducting a speech test for a subject using the subset of the pool of words.

2. The method of claim 1, wherein dynamically generating the subset of the pool of words comprises:
selecting the words from the pool of words such that an average phoneme distribution of the subset of the pool of words is within a predetermined threshold of a predetermined phoneme distribution value.

3. The method of claim 1, wherein dynamically generating the subset of the pool of words comprises:
implementing a search algorithm against a phoneme allocation table populated based upon the pool of words.

4. The method of claim 1, wherein dynamically generating the subset of the pool of words comprises:
determining an average phoneme distribution of the pool of words;
randomly selecting a word from the pool of words; and
accepting or rejecting the word for the subset of the pool of words based upon the average phoneme distribution of the pool of words and phonemes of the word.

5. The method of claim 1, further comprising:
compiling the pool of words from words with a similar phonetic structure.

6. The method of claim 1, wherein conducting the speech test for the subject comprises:
playing an audio signal of a word from the subset of words;
presenting, via a user interface, a visual indication of the word from the subset of words and a visual indication for each of a plurality of alternative words; and
receiving, via a user input device configured to interact with the user interface, a selection of the visual indication of the word from the subset of words or one of the visual indications for one of the plurality of alternative words.

7. The method of claim 6, further comprising:
determining the plurality of alternative words based upon a difficulty level for the speech test.

8. The method of claim 7, further comprising:
determining the plurality of alternative words based on an average phonetic distance of each of the plurality of alternative words from the word from the subset of words.

9. The method of claim 6, wherein conducting the speech test comprises:
presenting, via the user interface, a visual indication for the subject to indicate via the user input device that the audio signal of the word from the subset of words does not correspond to the visual indication of the word from the subset of words or any of the visual indications of the plurality of alternative words.

10. The method of claim 1, further comprising:
scoring the speech test.

11. The method of claim 10, wherein scoring the speech test comprises:
determining a discrimination loss for the subject or determining a sensitivity loss for the subject.

12. (canceled)

13. The method of claim 1, wherein conducting a speech test comprises:
presenting perceptually balanced recordings of the subset of the pool of words.

14. The method of claim 13, wherein presenting the perceptually balanced recordings of the subset of the pool of words comprises:
presenting the perceptually balanced recordings of the subset of the pool of words at substantially similar volume levels, or
presenting the perceptually balanced recordings to achieve a predetermined speech recognition threshold value.

15. (canceled)

16. The method of claim 13, wherein presenting the perceptually balanced recordings of the subset of the pool of words comprises;
presenting the perceptually balanced recordings of the subset of the pool of words with background noise.

17. The method of any one of claim 16, wherein presenting the perceptually balanced recordings of the subset of the pool of words comprises:
determining a background noise level for each of the perceptually balanced recordings to achieve a predetermined speech recognition threshold value.

18. The method of claim 1, wherein dynamically generating, from the pool of words, a subset of the pool of words with a predetermined phonetic balance comprises:
obtaining data indicative of the pool of words;
applying a search algorithm to the data indicative of the pool of words to generate data indicative of the subset of the pool of words having a selected phonetic distribution; and
generating, from the data indicative of the subset of the pool of words, a test set of words for the speech test for the subject.

19. The method of claim 18, wherein the selected phonetic distribution is a phonetic distribution within a threshold of the predetermined phonetic distribution.

20. The method of claim 18, further comprising:
generating, for each word of the test set of words, alternative words to be presented to the subject during the speech test.

21. The method of claim 20, wherein generating the alternative words comprises:
dynamically generating the alternative words based upon phonetic features of the word of the test set of words for which the alternative words are generated.

22. The method of claim 20, wherein dynamically generating the alternative words comprises:
dynamically generating the alternative words based upon a predetermined difficulty level for the speech test.

23. The method of claim 21, wherein dynamically generating the alternative words comprises:
selecting the alternative words based upon a level of phonetic similarity to the word of the test set of words for which the alternative words are generated.

24. The method of claim 18, further comprising:
administering the speech test to the subject using the test set of words.

25. The method of claim 24, wherein administering the speech test to the subject using the test set of words comprises:
generating a series of audio signals for presentation to the subject such that audio signals presented to the subject during the speech test are perceptually balanced.

26. The method of claim 25, wherein generating the series of audio signals includes selecting audio signals of varying volume levels.

27. The method of claim 25, wherein generating the series of audio signals includes generating audio signals with varying levels of background noise.

28.-31. (canceled)

32. One or more non-transitory computer readable storage media comprising instructions that, when executed by a processor, cause the processor to:
obtain a predetermined pool of words;
generate, from the predetermined pool of words, a subset of the pool of words having a predetermined phonetic distribution; and
generate, from the subset of the pool of words, a test set of words for administration of a speech test to a subject.

33. The one or more non-transitory computer readable storage media of claim 32, wherein the phonetic distribution of the subset of the pool of words is within a threshold of a predetermined phonetic distribution.

34. The one or more non-transitory computer readable storage media of claim 32, wherein the predetermined pool of words have a predetermined phonetic distribution, and wherein the phonetic distribution of the subset of the pool of words is within a threshold of the predetermined phonetic distribution of the predetermined pool of words.

35. The one or more non-transitory computer readable storage media of claim 32, further comprising instructions that, when executed by the processor, cause the processor to:
generate, for each word of the test set of words, alternative words to be presented to the subject during the speech test.

36. The one or more non-transitory computer readable storage media of claim 32, wherein the instructions that, when executed by the processor, cause the processor to generate the alternative words comprise instructions to:
dynamically generate the alternative words based upon phonetic features of the word of the test set of words for which the alternative words are generated.

37. The one or more non-transitory computer readable storage media of claim 32, wherein the instructions that, when executed by the processor, cause the processor to generate the alternative words comprise instructions to:
dynamically generate the alternative words based upon a predetermined difficulty level for the speech test.

38. The one or more non-transitory computer readable storage media of claim 36, further comprising instructions that, when executed by the processor, cause the processor to:
select the alternative words based upon a level of phonetic similarity to the word of the test set of words for which the alternative words are generated.

39. (canceled)
